(19) United States
(12) Patent Application Publication
(10) Pub. No.: US 2018/0350260 A1
(43) Pub. Date: Dec. 6, 2018

(54) SYSTEM AND METHOD FOR PROVIDING COGNITIVE STIMULATION WHICH IS REFLECTIVE OF LIFE EXPERIENCES

(71) Applicants: Shabbir Amanullah, London (CA); Heather Puhr-McNinch, Lakewood Ranch, FL (US)

(72) Inventors: Shabbir Amanullah, London (CA); Heather Puhr-McNinch, Lakewood Ranch, FL (US)

(21) Appl. No.: 15/990,745

(22) Filed: May 28, 2018

Related U.S. Application Data
(60) Provisional application No. 62/512,295, filed on May 30, 2017.

(51) Int. Cl. G09B 19/00

(52) U.S. Cl. CPC G09B 19/00; A61M 2021/005

(57) ABSTRACT

A system and method for providing cognitive stimulation which is reflective of life experiences is provided. The system includes one or more interactive module(s) reflective of life experience(s) and/or exposures.

### CROSS-REFERENCE TO RELATED APPLICATIONS

This application claims priority to U.S. Provisional Application Ser. No. 62/512,295 filed May 30, 2017, the contents of which are hereby incorporated by reference.

### FIELD OF INVENTION

This invention pertains to the field of cognitive stimulations and in particular, to a system and method for providing cognitive stimulation which is reflective of life experiences.

### BACKGROUND

Evidence supports an association with hospitalization and cognitive decline in elderly patients. A number of factors are thought to play a role and include but are not limited to medications, stress, depression and delirium (see for example, Neurology, 2012, 78:950-956; Am J Geriatr Psychiatry. 2014 May; 22(5): 465-480.). In addition, lack of cognitive stimulation may be associated with cognitive decline and cognitive stimulation may prevent such decline.

Hospital settings and long-term care facilities often lack cognitive stimulation. While technology, such as the use of tablets and game systems, may provide such stimulation, the use of such technology may present challenges for the elderly because many are uncomfortable with such technology and/or lack exposure to such technology. As a result, the use of such technology may cause anxiety and/or frustration and thereby by result in a negative experience for such elderly individuals.

Accordingly, there is a need in the art for cognitive stimulation which is reflective of the exposures and experiences of elderly patients.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An objection of the present invention is to provide a system and method for providing cognitive stimulation which is reflective of life experiences. In accordance with an aspect of the present invention, there is provided a system for providing cognitive stimulation and/or physical activity, said system comprising one or more module(s) reflective of life experience(s) and/or exposures. In accordance with one embodiment of the invention, the one or more module(s) comprises one or more component(s) configured to engage one or more sense(s) and/or skill(s).

In accordance with another aspect of the invention, there is provided a method for cognitive stimulation and/or providing physical activity comprising allowing an individual to interact with the system of the invention.

### BRIEF DESCRIPTION OF THE FIGURES

Embodiments of the invention will now be described, by way of example only, by reference to the attached Figures, wherein:

FIG. 1 provides a drawing of a front view of one embodiment of the system of the invention.

FIG. 2 provides an alternative view of the embodiment of FIG. 1.

FIG. 3 provides an alternative view of a part of the embodiment of FIG. 1.

FIG. 4 provides an alternative view of a part of the embodiment of FIG. 1.

FIG. 5 provides an alternative view of a part of the embodiment of FIG. 1.

### DETAILED DESCRIPTION OF THE INVENTION

The present invention provides systems and methods for maintaining brain activity and/or for providing cognitive stimulation and/or physical activity to individuals including but not limited elderly individuals, individuals in long term care, and individuals having or predisposed to cognitive decline. In particular, the present invention provides a system comprising one or more interactive module(s) or structure(s) which is reflective of life experiences and/or exposures. Also provided are methods which utilize the system of the invention to provide cognitive stimulation and/or physical activity.

The present invention is based on the recognition that cognitive stimulation may prevent cognitive decline in aging populations. Such stimulation is often absent in hospitals and continuing/long-term care facilities. While technology (including for example tablets, i.e. iPads and electronic game systems) may provide such stimulation, the use of technology may present challenges for certain individuals. For example, individuals who are uncomfortable with such technology and/or lack exposure to such technology, including but not limited to elderly individuals This may be further exacerbated in individuals which have difficulty with new learning, including for example, individuals with cognitive defects. It is further recognized that physical activity may slow physical decline in aging populations and maintaining physical activity in a hospital or long-term care facility may be challenging.

Accordingly, the present invention provides a system comprising one or more interactive module(s) or structure(s) which is reflective of life experiences and/or exposures which provides cognitive stimulation and/or physical activity. The module(s) and structure(s) can be configured to be interchangeable and/or readily modified. In some embodiments, modules and supporting structures have a “plug and play” configuration.

The system may also include be configured to allow users to return to previously started simulations thereby allowing users to gradual build up on previous exercises. In some embodiments, individual user information including records and data from sessions is stored in either system memory or removable memory such as a thumb drive.

In some embodiments, the module(s) or structures(s) can include refillable or replaceable comments including for example can include scent cartridges, pads of paper; paints, inks etc.

The systems of the invention can be configured for use in cognitive stimulation therapy and accordingly, in some embodiments include modules that allow for the assessment of user's interaction with the system. Accordingly, the system may include assessment modules. In some embodiments, assessment modules will record user activity and optionally maintain records of user activity. Optionally, the assessment modules include one or more cameras.

The system of the invention can be optionally configured to compare activities from different user's sessions to facilitate identification of changes in condition and optionally alert caregivers when significant changes in user's activity occur.

In some embodiments, the system includes one or more touch screens. Optional, the touch screen will be a large touch screen.

Life Experiences and/or Exposures

The module(s) are reflective of life experience(s) and/or exposures. The life experiences and/or exposures may be generic life experiences and/or exposures; may be tailored to one or more specific group(s) of individuals (such as a particular generation or age group (for example elderly individuals); particular profession(s), cultural group(s) or occupation(s) (non-limiting examples include farmers, miners, cooks and homemakers), particular activity (sports) or may be tailored to a particular individual (personalized). The life experiences and/or exposures may be from the past; the present; or future (i.e. potential experience and/or exposures). In certain embodiments, each module is themed to a particular era, occupation and/or daily activity.

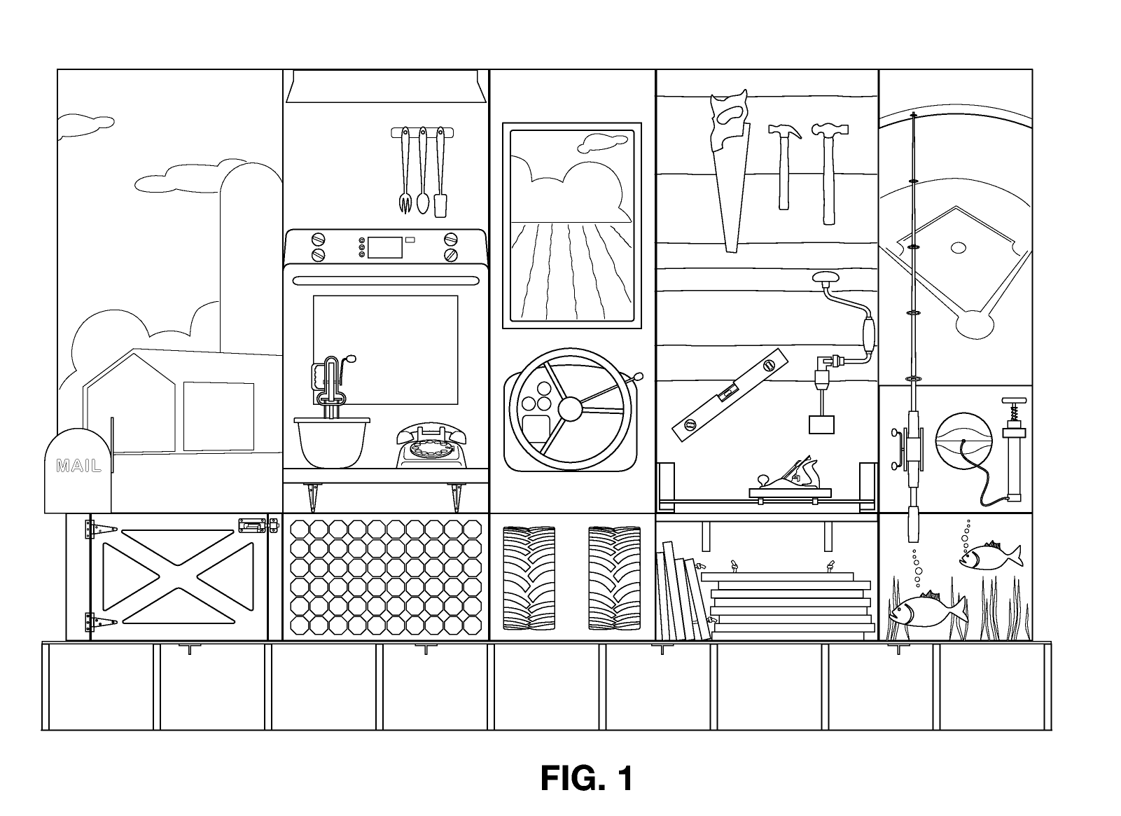

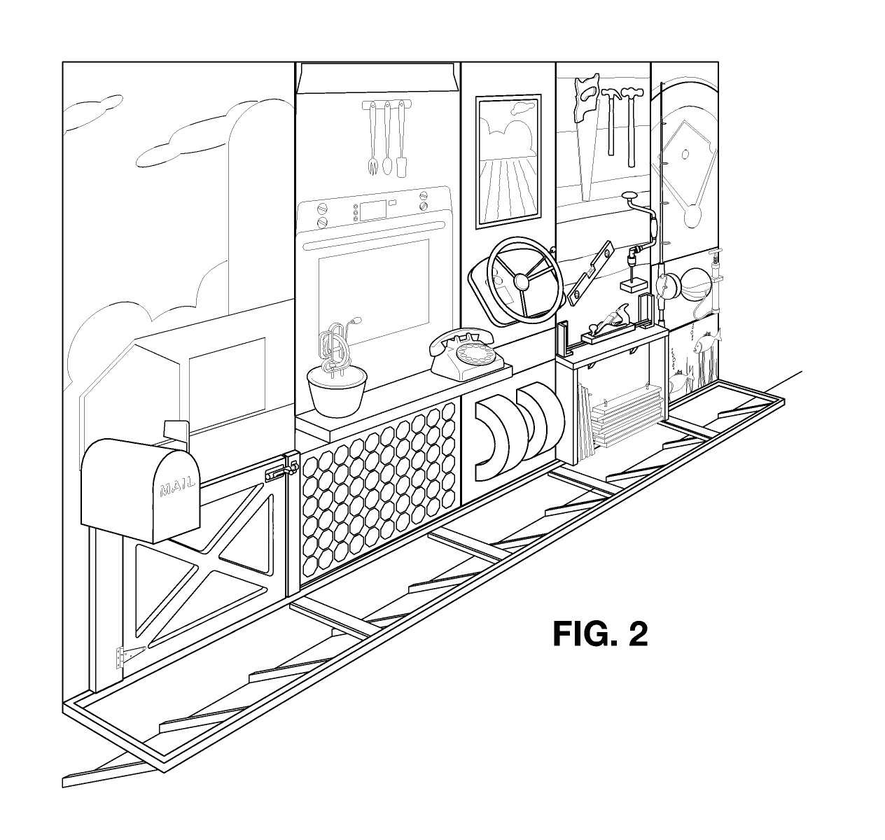

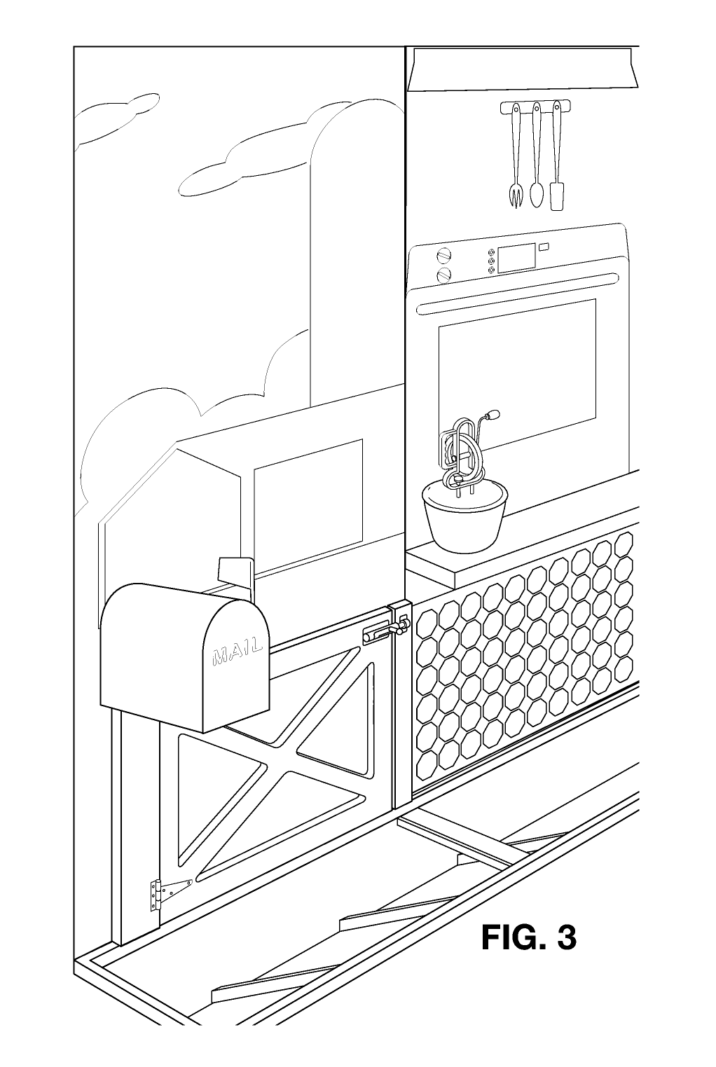

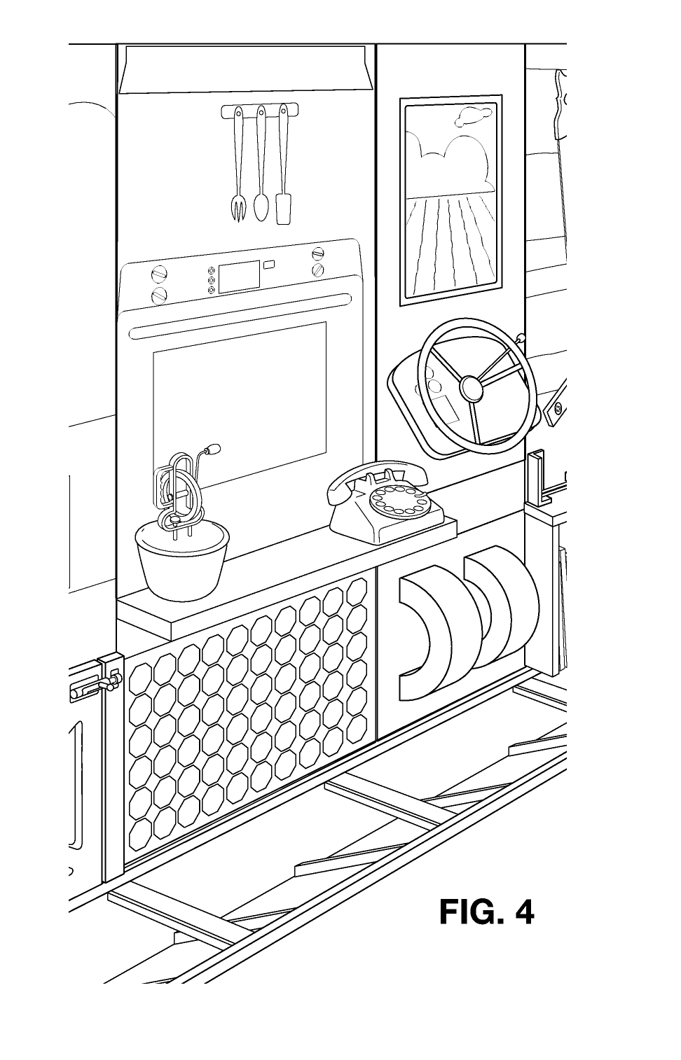

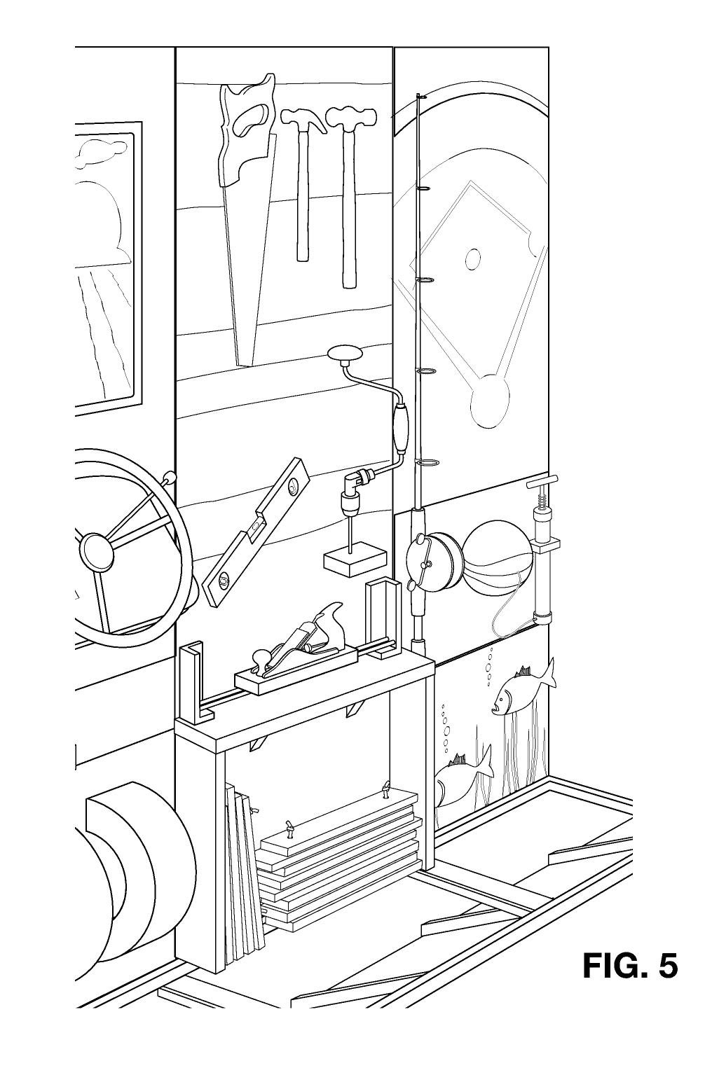

As an illustrative example only (see FIGS. 1 to 5), referring to FIG. 1 (from left to right) the system may include a module tailored to an individual whose past occupation was a dairy farmer, a second module may be tailored to an individual who was a housewife in the 1950's; and a third module may be tailored to a wheat farmer, a fourth module may be tailored to an individual whose past occupation was a carpenter and a five module may be tailored to a sports fan. The module tailored to the dairy farmer may include a mural of a farm yard which includes a barn, silo and cows, a mailbox which opens, a gate which opens and locks, a cow hide sample. The module tailored to a 1950s house wife may be for example kitchen themed and include a counter, kitchen utensils such as a hand mixer that is functional and a rotary dial phone which plays a recording. The module tailored to the wheat farmer may be designed to simulate driving a tractor through a field and include, for example, tractor components such as a steering wheel, gages, tire treads and images or video relating to a farm environment such as a field and/or a recording of sounds from a farm environment. The module tailored to a carpenter may include a woodshop theme mural and include wood working tools. The module tailored to a sports fans may include a mural of a sports stadium and include various sporting goods, such as a basketball which may be pumped up with air, a fishing rod which a reel that spins.

Other themes may include stores (such as grocery store), post office, other exterior urban or rural scenes (for example, nature scene, a street scene, a sports scene, exterior scene with a mailbox, gate and knocker or door bell), café or restaurant.

The modules may also include personal experiences. For example, the module may play personal recordings, such as home videos and/or audio recordings of friends or family members. This may allow an individual with cognitive decline to remain familiar with their home, friends and family members while in a hospital or continuing care environment.

In some embodiments, the system is configured to allow for input of personal recordings. Optionally, the recordings may be projected in a manner that gives the perception that scenes or images are three-dimensional or holographic. In some embodiments, the recordings may be interactive and responsive to user verbal cues. For example, a videographic companion could ask a user if they would like to go for a walk in the park or to town. The appropriate interactive video would play in response to the user's response by, for example, picking up key words.

In some embodiments, the system can be configured such that when a specific program or elements is activated other components are also activated. For example, when videos representative of a walk are activated a treadmill could be activated. Further, when the video appears to show an uphill walk, treadmill incline is adjusted to mimic walking uphill. The interaction of various modules and/or components can enhance the experience such that it feels more real. For example, a user could you the system to guided walking tour of the nursing or residential home they will move into, or indeed of their own homes to improve home return.

Images, videos are other sensory experiences are optionally adjusted at a speed or pace consistent with the walking pace of the user.

In some embodiments, the system includes one or more cameras to records patients response to the stimulation to help assess mobility and/or balance issues or environmental factors that trigger user stress or negative response. In such embodiments, the system may be equipped with gait recognition software.

In some embodiments, systems with one or more cameras include software that can enhance the patients experience by projecting their image for example in dance steps or yoga/Tai chi/relaxation exercises.

The modules may also include future (i.e. potential experiences and/or exposures). For example, a module may be reflective of a future nursing home or retirement home environment. In such cases, the module may play a video of the layout of the home thereby providing familiarity to the individual prior to placement in the home. A system which further includes a treadmill in combination with such videos may be used to simulate walking through the home.

Components which Stimulate Senses/Skills

The interactive module(s)/structure(s) include components which stimulate one or more of the senses including but not limited to visual, olfactory auditory and tactile; motor skills (gross motor and/or fine motor skills) or combinations thereof and facilitate the development and/or retention of skills including motor skills and cognitive skills.

In certain embodiments, the interactive module/structure comprises one or more components which provide visual stimuli. The visual stimuli may be specifically associated with the individual or generic. In certain embodiments, the auditory stimuli are reflective of life experience(s) and/or exposures. Exemplary visual components include but are not limited to images (including but not limited to digital images, murals, photographs, pictures and paintings), patterns (including for example wallpaper or tile mosaic patterns), textured surfaces (including but not limited to material, fur, brick, stone, tile, stucco, carpet, and paneling such as wood paneling), visual recordings, holograms, video, objects (including but not limited tools, utensils and household items).

In some embodiments, the visual stimulation includes eye tracking and/or coordination exercises. Optionally, users or caregivers can initiate eye movement exercises and/or eye relaxation exercises.

In certain embodiments, the interactive module/structure comprises one or more components which provide auditory stimuli. The auditory stimuli may be specifically associated with the individual or generic. In certain embodiments, the auditory stimuli are reflective of life experience(s) and/or exposures. Exemplary auditory components include but are not limited to recordings of voices (including but not limited to friends and relatives and other familiar voices), music, nature sounds, animal sounds, environmental, industrial, and commercial sounds.

In some embodiments, the auditory stimuli may include relaxation music and guided meditation and mindfulness exercise. The auditory stimuli can also include religious programs such as hymns, prays including Christian prayers, Muslim prayers, Jewish verses from the Torah, Hindu chants and Buddhist prayers. Optionally, religious stimuli can be provided in accordance with the liturgical calendar, or other calendars that mark important religious festivals.

In certain embodiments, the interactive module/structure comprises one or more components which provide tactile stimuli. The tactile stimuli may be specifically associated with the individual or generic. In certain embodiments, the auditory stimuli are reflective of life experience(s) and/or exposures. Exemplary tactile components include but are not limited to utensils, tools, household items, textured surfaces such as wood, fabric and fur (including simulated companion animals). Tactile stimuli can be on surfaces reachable by the user's hands, feet, face, legs, arms or other body parts. For example, in the system can include carpet, stimulated grass or other surfaces the user could walk across.

In certain embodiments, the interactive module/structure will include a number of different touch related products from velvet and cotton to steel and stones.

In certain embodiments, the interactive module/structure comprises one or more components which stimulate motor skills. The components may stimulate small motor and/or gross motor skills. The components may include for example tools (such as carpentry tools, garden tools), utensils (such as kitchen or writing utensils), household items (such as phone, record player, radio, mailbox), sporting equipment.

In certain embodiments, the system includes one or more touch screens. In such embodiments, the system may further include accessories usable with touch screens, for example, capacitive pens or paintbrushes that allow for the user to feel like they are using traditional pens or paintbrushes.

In certain embodiments, the system is configured to allow a user to solve puzzles, do simple tests including color matching and match/complete sentences. Optionally, the system is updatable to allow for new software or applications to facilitate development and testing of cognitive skills.

In certain embodiments, the system further comprises an exercise machine such as a treadmill. The treadmill is optionally configured to test for balance by having tilt and/or incline functions. In certain embodiments, the treadmill will be configured to be folded or otherwise stored when not in use.

Use of a treadmill may stimulate gross motor skills associate with gait and/or provide physical activity. In addition, it is known in the art that physical activity may be beneficial in preventing cognitive decline. For example, walking through a field, a nature trail or city may be simulated by including a module with a treadmill and a monitor playing a video of a field, nature trail or city street while the treadmill operates.

In certain embodiments, the components providing sensory stimuli may be responsive to input or may change particular stimuli based on a schedule. For example, a touch screen may be used and particular stimuli selected by the individual or caregiver; the stimuli may be activated by a sensor (such as a motion or optical sensor), the stimuli may require a physical action (such as turning a steering wheel or dialing or picking up a phone) or may be on a timer.

Construction and Design

The invention provides a system comprising one or more interactive module(s) or structure(s) which is reflective of life experiences and/or exposures which provides cognitive stimulation and/or physical activity.

In certain embodiments, each module of the system is a separate physical structure. For example, each module may be a panel comprising the components which may be free standing or fixed to an underlying structure such as a wall. Appropriate materials for the construction of panels for use in the construction of the system are known in the art and include wood or composite materials.

In other embodiments, the modules are spatially separated groupings of components on a wall or panel which may be attached to an underlying structure such as a wall or is free standing. The free standing structures may optionally include wheels (such as lockable wheels) to allow for easy transportation.

In certain embodiments, the system comprises interchangeable modules and/or components. This may allow the system to be modified to reflect the cognitive level, physical ability and/or life experiences. This also allows for replacement of damaged modules or components. For example, a module for individuals with a higher cognitive level may allow for more complex interactions. A module for more mobile individuals may include components which require the individual to reach up (stretch) or bend down and may also include a treadmill thereby providing physical activity while a module for limited mobility individuals may be wheel chair accessible. In other embodiments, the module may be repositioned. For example, may be elevated or lowered. In certain embodiments, the components on the module may be moved. In certain embodiments, the components are attached to the module with a tether thereby allowing movement of the component but preventing components being misplaced or harm coming to individuals by dropping the components. For example, tools or equipment such as a fishing rod may be tethered such that they may be picked up and handled.

In certain embodiments, the modules and/or components thereof are reversibly affixed to the underlying the structure. In specific embodiments, the modules are mounted on a rail system which optionally allows for installation/removal in a slide on-off manner.

In some embodiments, the mounting system is designed to allow panels to be slid into place or to be installed by an individual. The mounting system is optionally configured to provide for quick installation.

In some embodiments, the mounting system may include tracking.

In certain embodiments, background images of the module are on interchangeable screens. In certain embodiments, the components are inserted in slots and components may be added or removed as necessary.

In certain embodiments, the structure or one or more modules may include a cover. The cover may be used to limit access to the structure or modules.

Methods

The present invention also provides methods for cognitive stimulation and/or physical activity by allowing an individual to interact with the system of the invention. In certain embodiments, individuals are allowed to freely interact with one or more modules (i.e. free play). In certain embodiments, the interaction is accessed and modifications to one or more modules are made in response to the interaction. In certain embodiments, prior to the interaction, the system is configured such that the system reflects the individual user life experiences and/or exposures. The information used to modify the system may be obtained from a variety of sources including but not limited to directly from the individual, family members, the patient files or records, or public records. The individuals or users may be patients in a hospital setting, residents of a long-term care or retirement home. The individuals may be elderly, have cognitive impairment including mild cognitive impairment or dementia including but not limited to Alzheimer's disease.

The systems and methods may also be used to provide cognitive stimulation for other individuals, including individuals with cognitive impairments, autism, aphasia patients, and stroke patients.

1. A system for providing cognitive stimulation and/or physical activity, said system comprising one or more module(s) reflective of life experience(s) and/or exposures.

2. The system of claim 1, wherein each of said one or more module(s) comprises one or more component(s) configured to engage one or more sense(s) and/or skill(s).

3. A method for cognitive stimulation and/or providing physical activity comprising allowing an individual to interact with the system of claim 1 or 2.
